(19) United States
(12) Patent Application Publication
(10) Pub. No.: US 2012/0265055 A1
(43) Pub. Date: Oct. 18, 2012

(54) TUBULAR FEEDING DEVICE HAVING SHAPEABLE DISTAL END

(76) Inventors: Jeffry S. Melsheimer, Springville, IN (US); Nathaniel A. Irwin, Bloomington, IN (US); Amro Kamel, Bloomington, IN (US)

(21) Appl. No.: 13/087,956

(22) Filed: Apr. 15, 2011

(51) Int. Cl. A61M 39/08; A61B 8/12

(52) U.S. Cl. 600/424; 604/528

(57) ABSTRACT

A tube for insertion into a body passageway of a patient includes a generally elongated tubular member having a proximal portion, a distal portion, a lumen extending between the proximal portion and the distal portion, and at least one aperture at the distal portion sized and positioned for passage of fluid material therethrough from the lumen to a target area in the body passageway. The tubular member is structured such that the distal portion is selectively movable between the generally elongated configuration and a shaped configuration. A length of the distal portion is provided with one or more echogenic members disposed therealong such that the distal portion length is visible under ultrasound visualization when in the shaped configuration.

### BACKGROUND

1. Technical Field

The present invention relates to tubular medical devices. More particularly, the invention relates to a tubular feeding device having a shapeable distal end for enhanced visualization under medical imaging.

2. Background Information

Patients for whom normal ingestion of food becomes difficult or impossible may require placement of a feeding tube to assist in providing their nutritional needs. For some individuals, such as comatose patients, stroke victims, or those with a compromised gastrointestinal (GI) tract, this may require placement of a tube that is introduced percutaneously into the stomach for delivery of nutritional products directly into the stomach. Such tubes for delivery of nutritional products into the stomach are generally referred to as gastrostomy tubes, or “G”-tubes.

In some situations, feeding a patient through a G-tube positioned in the stomach can be problematic. For example, the presence of certain congenital abnormalities in the patient's stomach may obstruct proper placement of the tube. Suitable placement may also be hindered when the patient exhibits severe gastric reflux and/or a high rate of aspiration. In these and other situations, nutritional targets may not be attained at a satisfactory rate through G-tube feeding. In such patients, feeding may often be accomplished at a suitable rate by inserting a feeding tube, sometimes referred to as a jejunal tube, or a “J”-tube, directly into the jejunum of the patient. The J-tube bypasses the stomach, thereby avoiding many congenital abnormalities, and decreasing the risk of gastric reflux and/or aspiration. The J-tube often provides better success in delivering nutrients than a G-tube, and allows the nutrients to be delivered and absorbed more rapidly.

Notwithstanding the foregoing, however, there are some difficulties associated with the use of jejunal feeding tubes. For example, due to the generally offset position of the jejunum relative to the stomach, it is often difficult to properly direct the distal end of a J-tube into the jejunum. J-tubes are typically very flexible, which contributes to the difficulty in directing the tubes to the desired area. In addition, once positioned, J-tubes are subject to dislodgement.

In view of the difficulties encountered in placing such tubes in the jejunum, radiographic imaging techniques, e.g., x-ray, are utilized to verify proper placement of such tubes. As health care workers must transport that patient to the radiology facility to obtain the x-ray, this technique increases the cost and complexity of the feeding tube placement. In addition, the use of radiographic imaging exposes the patient to radiation. If the x-ray indicates that insufficient placement was achieved, then the verification process must be repeated following another attempt at placement. This adds still more cost and complexity to the procedure, and further increases the amount of radiation to which the patient is exposed.

Ultrasound visualization is an alternative imaging modality. Ultrasound visualization has favorable characteristics in that it can be performed at the bedside, and it eliminates radiation exposure to the patient. However, the use of ultrasound visualization can be problematic if a volume of air/gas is present between the ultrasound transducer head and a structure being visualized. The gastrointestinal tract has a generally “pipe-like” configuration along much of its length. As the feeding tube advances along the GI tract during insertion, it may track the posterior intestinal wall of this tract, leaving an air gap within the intestinal lumen along the anterior wall. Since the transducer head is positioned on the side of the anterior wall, the presence of the air gap inhibits optimal visualization of the feeding tube under ultrasound.

It would be desirable to provide a feeding tube suitable for placement in the jejunum of the patient, wherein the feeding tube is structured in a manner such that the position of the feeding tube may be viewed by means readily available at the patient's bedside, and by means that do not expose the patient to harmful radiation.

### BRIEF SUMMARY

The present invention addresses the shortcomings in the prior art. In one form thereof, a tube is provided for insertion into a body passageway of a patient. The tube includes a generally elongated tubular member having a proximal portion, a distal portion, a lumen extending between the proximal portion and the distal portion, and at least one aperture at the distal portion sized and positioned for passage of fluid material therethrough from the lumen to a target area in the body passageway. The tubular member is structured such that the distal portion is selectively movable between the generally elongated configuration and a shaped configuration. A length of the distal portion comprises an echogenic capability such that the distal portion length is visible under ultrasound visualization in the shaped configuration.

In another form thereof, a method is provided for positioning a feeding tube in the jejunum of a patient. A distal end of a feeding tube is inserted into an oral cavity of a patient. The feeding tube comprises a generally elongated tubular member having a proximal portion, a distal portion having an echogenic surface, a lumen extending between the proximal portion and the distal portion, and at least one aperture at the distal portion for passage of fluid material from the lumen to the jejunum. A stiffening member extends along the tubular member proximal portion and distal portion. The tubular member is selectively maneuverable between the generally elongated condition when the stiffening member extends therealong, and a shaped condition along the distal portion when the stiffening member is withdrawn from the distal portion. The feeding tube distal end is advanced through the stomach of the patient such that the distal portion of the generally elongated tubular member extends into the small intestine of the patient. The stiffening member is withdrawn from the distal portion such that the distal portion maneuvers into the shaped condition. A placement of the shaped distal portion is the viewed via ultrasound visualization of the echogenic surface.

In yet another form thereof, a tube is provided for insertion into a body passageway of a patient. A generally elongated tubular member has a proximal portion, a distal portion, a pair of lumens extending between the proximal portion and the distal portion, and at least one aperture along a length of the tubular member sized and positioned for passage of fluid material therethrough from a first lumen to a target area in the body passageway. The proximal portion of the tubular member has a higher stiffness, and the distal portion of the tubular member has a lower stiffness. The distal portion has an echogenic material disposed therealong. A mandrel is slidably received in a second lumen. The mandrel has a stiffness less than a stiffness of the tubular member proximal portion, and greater than a stiffness of the tubular member distal portion. The mandrel is structured such that a distal length thereof has a tendency to assume a shaped configuration in an absence of restraint thereupon, whereby when the mandrel distal length is received along the tubular member proximal portion having the higher stiffness, the mandrel distal length has the generally elongated condition of the tubular member proximal portion. When the mandrel distal length is received along the tubular member distal portion having the lower stiffness, the mandrel distal length and the tubular member distal portion having the echogenic material disposed therealong assume the shaped configuration.

### BRIEF DESCRIPTION OF THE DRAWINGS

FIG. 1 is a side view of the distal portion of one embodiment of a feeding tube;

FIG. 2 is a transverse cross-sectional view taken along line 2-2 of FIG. 1;

FIG. 2A is a transverse cross-sectional view of an alternative embodiment of the feeding tube of FIG. 1;

FIG. 3 is a side view of a mandrel to be received in a lumen of the feeding tube;

FIG. 4 illustrates a segment of the distal portion of the feeding tube formed into a loop;

FIG. 5 illustrates an embodiment of a feeding tube including a suture for drawing the feeding tube into a looped configuration;

FIG. 6 illustrates a segment of the feeding tube of FIG. 5 drawn into a loop;

FIGS. 7-10 illustrate in sequence an insertion of the feeding tube of FIG. 1 into the jejunum, and visualization of the distal portion of the tube, according to one embodiment of the present invention;

FIG. 11 is a side view of another embodiment of feeding tube;

FIG. 12 is a transverse cross-sectional view of the feeding tube of FIG. 11 along line 12--12;

FIG. 13 is a longitudinal cross-sectional view of the feeding tube of FIG. 11;

FIG. 14 illustrates a mandrel to be received in a lumen of the feeding tube of FIG. 11;

FIG. 15 illustrates the mandrel of FIG. 14 when received in the lumen of the feeding tube;

FIG. 16 is a sectional view showing receipt of the mandrel in the feeding tube lumen as shown in FIG. 14, wherein the mandrel is advanced into the distal portion of the feeding tube; and

FIG. 17 illustrates a side view of the feeding tube when the mandrel is advanced as shown in FIG. 16.

### DESCRIPTION OF THE PREFERRED EMBODIMENTS

For purposes of promoting an understanding of the present invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the feeding tube, as well as the axial ends of various component features. The term “proximal” is used in its conventional sense to refer to the end of the feeding tube (or component thereof) that is closest to the operator during use. The term “distal” is used in its conventional sense to refer to the end of the feeding tube (or component thereof) that is initially inserted into the patient, or that is closest to the patient during use.

FIG. 1 is a side view of the distal portion of a feeding tube 10, according to an embodiment of the present invention. The proximal portion of the feeding tube is conventional, and need not be shown to achieve an understanding of the features of the present invention. Feeding tube 10 may be a jejunal feeding tube. Typically, a jejunal feeding tube is inserted into the jejunum via the oral cavity (nose or mouth). In the following non-limiting description, feeding tube 10 is a naso-jejunal feeding tube, that is, a feeding tube inserted via the nasal cavity. Those skilled in the art will appreciate that in an appropriate case feeding tube 10 may be inserted into the jejunum other than through the nasal cavity, e.g., through the stomach or through the mouth. In addition, feeding tube 10 need not necessarily be advanced such that the distal opening extends into the jejunum. Rather, in some instances, suitable results may be achieved if the distal end of the tube is positioned in portions of the small intestine other than the jejunum. It is generally preferred, however, that the distal end of the tube resides in the jejunum. In addition to providing nutritional products into the jejunum, tube 10 may also be used for delivering other fluid materials, such as drugs and/or contrast materials, to other locations in the alimentary canal.

Feeding tube 10 comprises an elongated tubular member 12 having a distal portion 14. Feeding tubes, such as naso-jejunal tubes, are well known in the art, and tube 10 may be formed from any compositions commonly used and/or appropriate for such purposes. Polyurethane, silicone, polyurethane-silicone copolymers, and PVC are non-limiting examples of such compositions. Typically such tubes have a length of about 150-160 cm, and an outer diameter of between about 8 and 16 French (2.6 to 5.3 mm). Those skilled in the art will appreciate that the length and diameter of a feeding tube may be varied as desired to account for differences in patient size and anatomy.

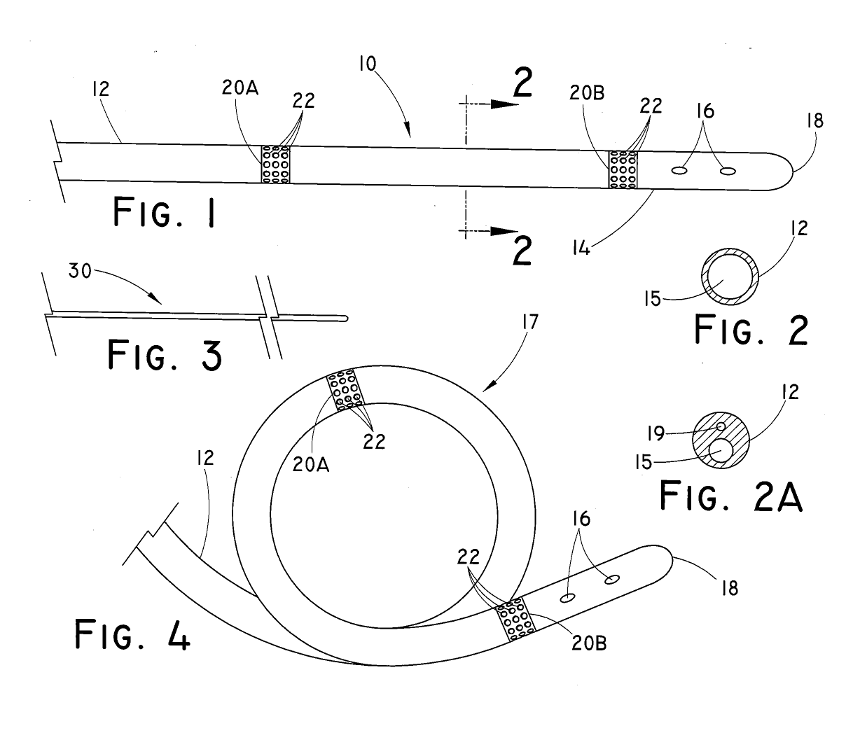

As shown in FIG. 2, elongated tubular member 12 has a lumen 15 lumen extending therethrough. Lumen 15 is sized for transmission of a liquid product to the patient. Although tubular member 12 is shown with a single lumen extending therethrough, one or more additional lumens may be provided if desired. FIG. 2A illustrates an embodiment wherein an auxiliary lumen 19 is provided in addition to main lumen 15. When one or more auxiliary lumens are present, typically the main lumen, e.g., lumen 15, has a relatively large diameter, and the other lumen(s) have a smaller diameter. When present, the auxiliary lumen(s) may be utilized for receiving a stiffening member, as discussed herein.

Those skilled in the art will appreciate that additional auxiliary lumens may be provided if desired for other known purposes. Such additional lumens could be used, e.g., to provide an additional fluid source, such as a liquid medication in addition to the liquid nutritional products, and/or to monitor various pressures or functions within the patient's body. An alteration in the number of lumens may necessitate minor alteration in the features of the inventive tube as described herein; however those skilled in the art can readily make such alterations when following the teachings of the present invention.

As shown in FIG. 1, tube distal portion 14 comprises one or more feeding tube side ports, or apertures, 16 disposed along the distal portion of the tube. Side ports 16 provide openings through which nutritional products or other fluid material can exit the interior of the feeding tube, and enter the jejunum. Typically, the side ports are round or elliptical, and have dimensions well known in the art for such purposes (e.g., between about 2.5 and 4 mm maximum diameter). Those skilled in the art will appreciate that side ports of other configurations and dimensions may be substituted. Side ports 16 may be sequentially disposed along opposite sides of the feeding tube, e.g., at approximately 2 cm intervals. Alternatively, side ports 16 may be randomly distributed along tube distal portion 14 in any number, and in any arrangement, that permits passage therethrough of liquid or fluid products in a manner suitable for the intended use of tube 10. Although referred to herein as “liquid” or “fluid” products, the materials passing through the side ports may be of various consistencies and viscosity, and therefore, the “liquid” or “fluid” products may also include semi-solid portions, all as well known in the art.

Typically, tubular member 12 tapers to a closed distal end, or tip, 18. When the distal end is closed, all fluid material passes through side ports 16. In addition, a conventional guiding or tracking member (e.g., stiff wire guide, mandrel, or stylet) can be used to aid initial insertion of the tube if desired. In some instances, however, it may be preferred to maintain an open distal end to permit passage of liquid product therethrough. When the feeding tube has an open distal end, the presence of side ports 16 is optional.

Those skilled in the art will appreciate that the feeding tube may include additional features well known in the art. For example, the outer surface of the elongated tubular member may be provided with a series of fin-like projections along the distal portion thereof to enhance the advancement of the feeding tube into the jejunum via peristalsis. Feeding tubes having features that promote self-advancement by peristalsis are further described in, for example, U.S. Pat. Nos. 6,589,213 and 6,767,339, both incorporated by reference herein. Additionally, the feeding tube may be provided with a series of markings displayed at discrete locations along the length of the tubular member to monitor advancement of the tube into the jejunum.

It is sometimes difficult to direct the distal end of a tube to a target site within the anatomy of a patient that is off-set, or has a non-direct path, relative to an entry site. A feeding tube that is intended to be directed into the jejunum of a patient is an example of such a tube. Due to the difficulty in inserting tubes to such sites, it is generally desirable to verify the placement of the tube following insertion. When verifying the placement of a feeding tube in the jejunum, the normal procedure is to transport the patient to the radiology facility to obtain an x-ray. However, this procedure increases the cost and complexity of the feeding tube placement. In addition, the procedure exposes the patient to radiation.

Tube 10 is provided with an echogenic feature that enables the health care worker to observe in real time the location of the end of the tube in a body passageway. This echogenic feature enables tip confirmation to be carried out under ultrasound visualization at the patient's bedside. As a result, there is no need to transport the patient to another location for x-ray verification, and the patient need not be exposed to harmful radiation.

The echogenic feature may be imparted to the tubular member in a variety of ways. In the non-limiting embodiment shown in FIG. 1, the echogenic feature comprises one or more echogenic members, such as bands 20A, 20B, disposed along the distal portion of the elongated tubular member. The echogenic bands may be formed of a metal or metal alloy (e.g., stainless steel), and are structured in a manner to promote scattering and/or reflecting of ultrasound energy back to the ultrasound transducer head. If desired, the bands may be formed of radiopaque metals or alloys (e.g., palladium, tungsten, and platinum). In this case, in addition to having an echogenic surface for visualization by ultrasound, the bands can also be visualized under fluoroscopy if desired.

Echogenic bands 20A, 20B may be incorporated into tubular member 12 in a variety of ways. For example, the tubular member 12 may be stretched in longitudinal fashion to a smaller diameter, and the band may be inserted thereover. When the stretching is relaxed, tubular member 12 returns to its original diameter, and the band is snugly engaged therewith. Alternatively, respective bands 20A, 20B may be placed within the lumen of the tubular member, and the bands locked in place by melting the tubular member, e.g., during closure of the tubular member distal tip 18 when exposed to heat in a conventional tip-forming device. It is known to fashion bands or rings, such as echogenic bands 20A, 20B onto tubular members, such as catheters, and a skilled artisan can readily fashion a suitable technique for use herein. Tubular members having echogenic bands or sheaths applied thereto are known in the art for other purposes. Examples of catheters having echogenic bands or like enhancements include the ECHOTIP® ureteral catheter, and the ECHOTIP® Soft-Pass Embryo Transfer catheter, both available from Cook Medical of Bloomington, Ind.

The echogenic surface of bands 20A, 20B may comprise a series of irregularities, such as deformations 22, distributed along the exterior surface of the band. Deformations 22 are imperfections that are formed along the surface of the band in a manner that enhances the ability of the band to scatter and/or reflect the ultrasound energy. The deformations may be formed along the length of the band by well-known processes, such as media blasting, physical deformation, machining (e.g., knurling on a lathe), micro-dimpling, etc. Those skilled in the art will appreciate that there are many other ways of forming deformations in a substrate of a type that will result in the scatter and/or reflectance of ultrasound signals, and that may be substituted for the techniques described above. Deformations 22 should be formed in a manner such that they do not adversely affect the mechanical properties of the band in any material fashion.

The presence of the echogenic enhancement, such as deformations 22, causes ultrasound waves that contact the deformations to travel in multiple directions and in generally random fashion. The increase in scatter and/or reflection of the ultrasound waves enhances the temporal visualization of the tip of the feeding tube during ultrasound examination. By viewing the ultrasound signals created thereby, the health care worker may confirm proper tip location. Alternatively, if it is determined upon visualization that the desired tip location has not been achieved, the location of the tube may be adjusted on site.

Feeding tube 10 may be provided in combination with a stiffening member 30, such as a mandrel or a wire guide, as shown in FIG. 3. Mandrel 30 is sized to be received in a lumen of the feeding tube. In a preferred embodiment, the mandrel is sized to be received in main lumen 15 (FIG. 2) extending through the feeding tube. However, this arrangement is not critical, and the mandrel may alternatively be received in an auxiliary lumen that may be provided along the length of the feeding tube, such as lumen 19 in FIG. 2A. Mandrel 30 may be formed of a composition having sufficient stiffness, relative to a stiffness of the elongated tubular member 12, such that the tubular member is maintained in the elongated condition shown in FIG. 1 when the mandrel is received in the lumen. Typically, mandrel 30 will be formed of a metal or metal alloy, such as stainless steel or nitinol, and will extend substantially the length of the tubular member.

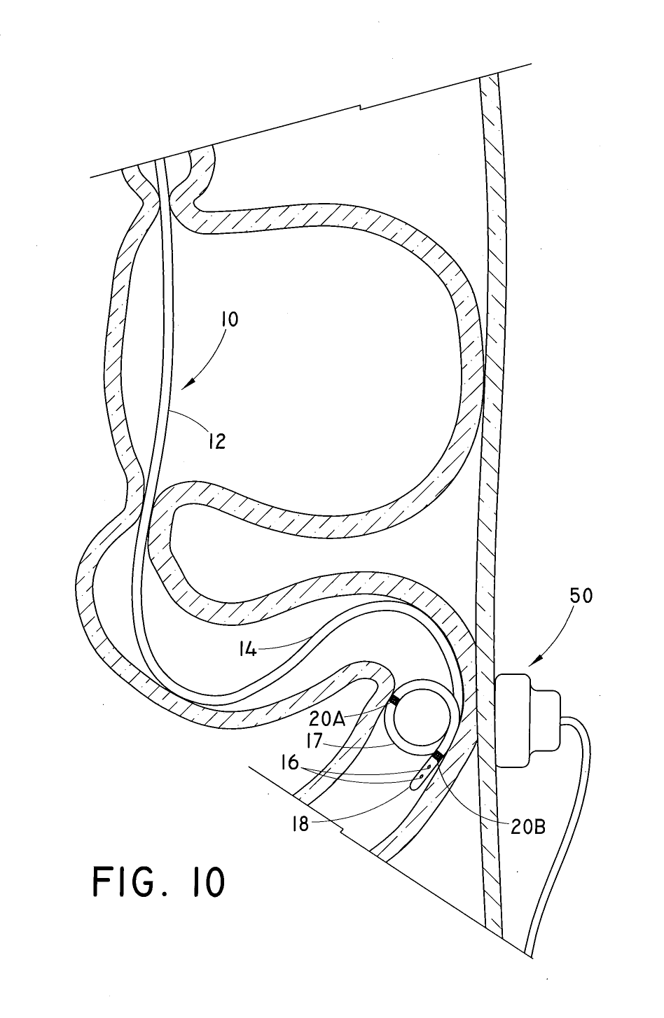

Tubular member 12 may be formed to have an internal memory, e.g., a shape memory or an elastic memory. In this manner, when mandrel 30 is withdrawn from the lumen, the distal portion 14 of the tubular member curls or is otherwise maneuvered into a pre-arranged, shaped configuration. The shaped configuration preferably includes at least one curve, and more preferably, comprises at least one loop or loop-like configuration 17 (collectively referred to herein as a “loop”), as shown in FIG. 4. Desirably, the internal memory of tubular member 12 is such that upon formation of loop 17, the loop has a diameter that substantially spans the inside diameter of the jejunum. See, e.g., FIG. 10.

Those skilled in the art are aware of many suitable processes for preparing a substrate to be capable of taking on a pre-arranged shaped configuration as described. One preferred way of treating a tubular member to return to a shaped configuration is by heat setting a specified distal length of the tubular member. This may be carried out, e.g., by placing the specified tubular member distal length in a heated glycerin solution to soften the polymer, and thereafter placing the heated portion of the tubular member in a suitably-shaped mold. Upon cooling, the designated portion of the tubular member takes on the desired configuration. In this manner, the tubular member can be temporarily straightened for insertion into a body opening, e.g., by insertion of the stiffening member into the tubular member lumen as described. Once the tubular member is advanced to the target site, the stiffening member is removed, and the tubular member will revert to the pre-arranged shaped configuration.

Any number of echogenic members can be applied to the tubular member, and the members can be spaced at varied lengths along the tubular member. In the non-limiting embodiment shown in FIGS. 1 and 4, two bands, or rings, 20A, 20B are positioned along a length of the tubular member. Preferably, the bands are spaced along the length of the tubular member in a manner such that they become generally diametrically opposed from each other once the stiffening member is removed, and the loop 17 reverts to its pre-arranged configuration. Those skilled in the art will appreciate that the heat set of tubular member distal portion 14 can be formed to define the loop as shown in FIG. 4, with the bands 20A, 20B at the opposing sides of the loop.

Although the embodiment shown in FIGS. 1-4 includes two bands spaced in a manner such that the bands diametrically oppose each other upon formation of the loop, other numbers and arrangements of bands may be substituted. For example, four bands may be positioned such that they are spaced, e.g., approximately 90 degrees from each other along the circumference of the loop.

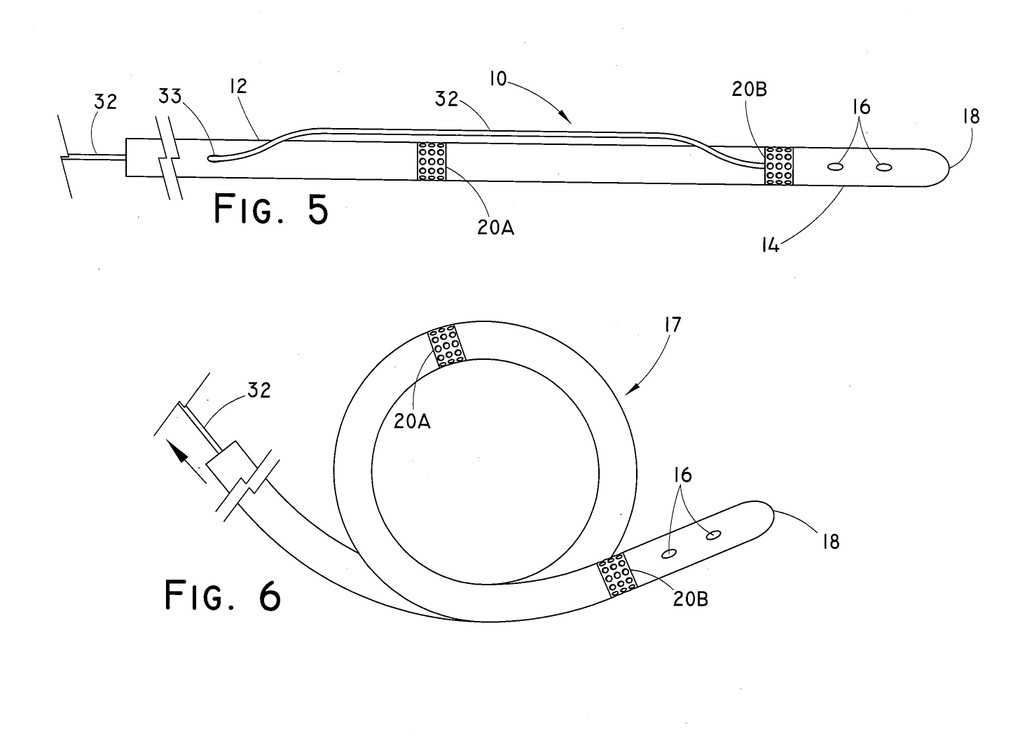

Although loop 17 has been described above as formed via a heat-set of the distal portion of the tubular member, those skilled in the art will appreciate that there are other suitable ways to achieve a desired shaped configuration for a designated length of a catheter or other tubular member. FIGS. 5 and 6 illustrate one such alternative. In this case, a feeding tube 10 comprises a tubular member 12 having a distal portion 14, one or more side ports 16, a closed distal end 18, and one or more echogenic members, such as bands 20A, 20B, as generally described above in the description of FIG. 1. A tension member 32, such as a suture, is arranged in a manner to enable distal end 14 to be drawn into a looped configuration. Tension member 32 may extend along a lumen of the tubular member to a side port 33, whereupon the tension member exits the lumen and extends along a length of the tubular member distal portion 14 to an attachment point. In FIG. 5, the attachment point comprises a space between band 20B and tubular member distal portion 14 that securely captures the distal end of the tension member, although those skilled in the art can readily fashion an alternative attachment point.

The proximal end of tension member 32 preferably extends beyond the proximal end of feeding tube 10 to enable easy grasping and drawing of the tension member in the proximal direction to form a desired shaped configuration, such as a loop configuration. When the desired shaped configuration is achieved, the tension member may be knotted, or locked in a suitable locking mechanism (not shown) to maintain the shaped configuration. Locking mechanisms suitable for use with tension members to lock a portion of a catheter or like device into a desired shaped configuration are well known in the art, and need not be further described. Such mechanisms are commonly utilized in connection with, e.g., drainage catheters and the like which are often drawn into a looped configuration and locked therein. One example of such a locking mechanism is provided in U.S. Pat. No. 5,399,165, incorporated by reference herein.

In one embodiment, the two techniques described above may be combined. That is, the tubular member may be heat set to achieve the looped configuration shown in FIG. 6, e.g., upon removal of a stiffening member, or upon grasping and drawing the tension member 32 in the proximal direction. The tension member may then be locked in place to secure the looped configuration.

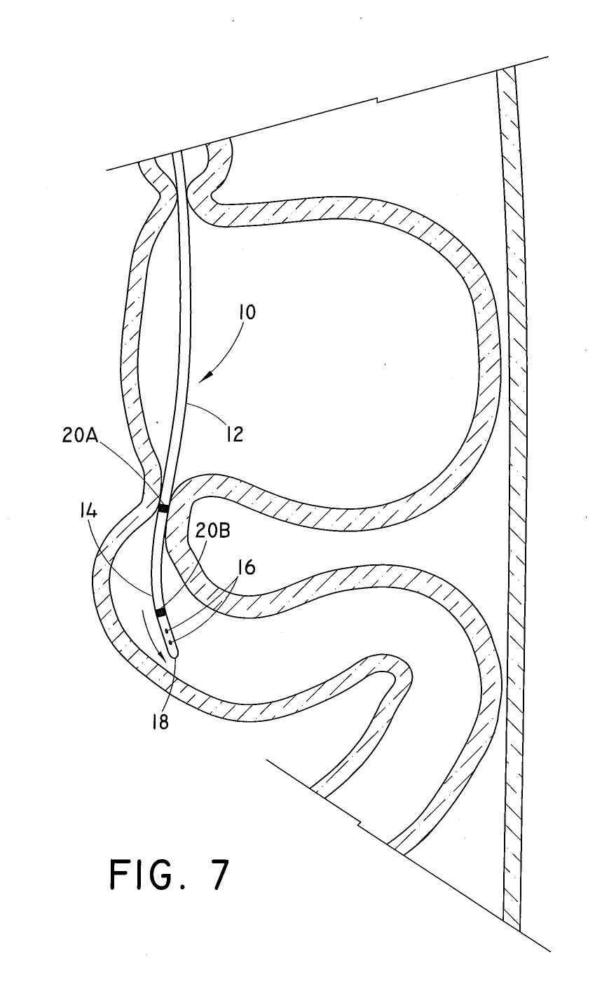

In the example depicted in FIGS. 7-10, a distal end of a tube is positioned in a body passageway of a patient. The feeding tube is a naso-jejunal feeding tube, and the distal tube end is advanced into the jejunum of the patient. In these figures, the cross-sectional diameter of the jejunum has been exaggerated relative to the internal space of the stomach in order to enable better visualization of the positioning of the feeding tube in the jejunum.

Initially, the distal end of tube 10 is inserted into an oral cavity of the patient (e.g., nose or mouth), and advanced about 50-70 cm into the stomach. As stated above, a guiding or tracking member (e.g., stiff wire guide, mandrel, stylet) may be inserted into the tube prior to placement to assist in insertion into the stomach. Insufflation and/or auscultation may be used to confirm the position of the distal tip of tube 10 in the stomach.

At this time, the feeding tube is advanced through the stomach of the patient such that the distal portion of the generally elongated tubular member extends into the small intestine of the patient. Typically, the distal end portion 14 of the feeding tube is advanced into the small intestine via peristaltic activity along the GI tract. FIG. 7 illustrates the position of distal portion upon entrance to the small intestine from the stomach. In cases of weak peristaltic activity, pharmacological agents may be used to increase this activity pursuant to well-known techniques. Reliance on peristaltic activity is optional, however, and other known means of directing a feeding tube to the jejunum may be employed, either in conjunction with peristalsis, or as an alternative to peristaltic activity.

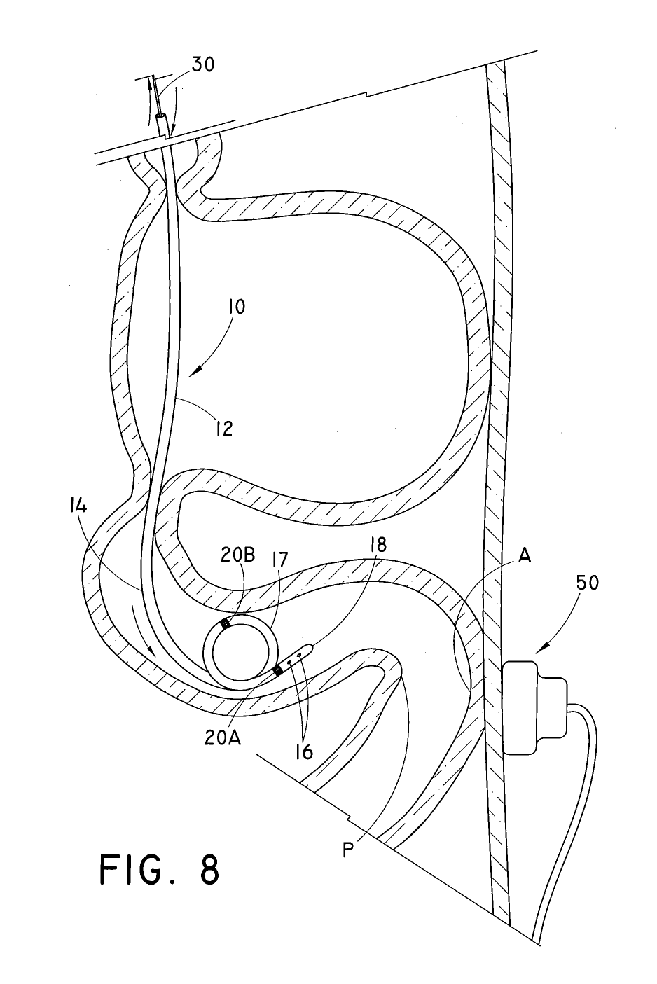

As shown in FIG. 8, distal portion 14 has been advanced a distance through the small intestine. If it is desired to determine whether distal portion 14 has advanced into the jejunum, stiffening member 30 can be withdrawn in the proximal direction, as shown by the arrow, such that distal portion 14 reverts to loop 17. A conventional ultrasound transducer head 50 is properly aligned, and an attempt is made to determine the position of feeding tube distal portion 14 via ultrasound visualization. Since the feeding tube has not been sufficiently advanced into the jejunum, a lengthy air gap is present along the interior of the small intestine between the ultrasound transducer head and the distal end of the feeding tube. The insufficiency of the ultrasound signal obtained thereby, and the accompanying ultrasound image obtained, inform the clinician that an unsatisfactory placement of the feeding tube distal end has been made. Thus, the feeding tube is further advanced in the small intestine.

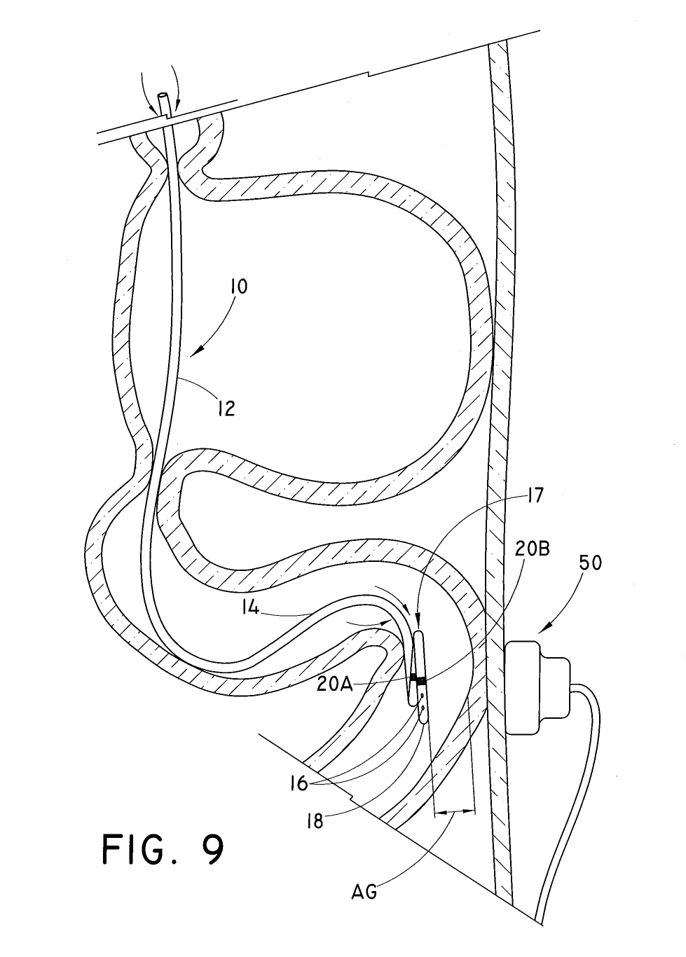

Following further advancement, the clinician may again investigate whether appropriate placement of the distal portion 14 has been achieved. As shown in FIG. 9, distal portion 14 has been advanced into the jejunum. However, in this arrangement, even though the distal portion 14 is in the jejunum, an adequate confirmatory image may still not be obtained under ultrasound visualization. Since, in this example, loop 17 is tracking the posterior jejunal wall, an air gap (AG) is still present between the feeding tube and the ultrasound transducer head 50 positioned along the anterior wall. As stated above, the presence of the air gap inhibits optimal visualization of the feeding tube under ultrasound.

In some cases, the clinician may assume that notwithstanding the lack of an optimal ultrasound image due to the air gap, appropriate placement has been attained. This assumption may be formulated from past experience in feeding tube placement, or alternatively, by monitoring the length of feeding tube that has been inserted (e.g., by viewing spaced markings along the length of the feeding tube). In order to minimize the possibility of an erroneous reading due to the air gap AG as shown in FIG. 9, the clinician may simply rotate the proximal end of the feeding tube as shown in FIG. 9. Upon rotation of the proximal end of the feeding tube, sufficient torque is transmitted through the length of the feeding tube to rotate distal portion 14 from the position shown in FIG. 9, to the position shown in FIG. 10. At this time, another ultrasound image is obtained. Since the air gap AG between the ultrasound transducer head 50 and the echogenic band 20B shown in FIG. 9 has been substantially eliminated in the arrangement shown in FIG. 10, the real-time ultrasound image of this arrangement indicates suitable placement of the distal end of the feeding tube in the jejunum. Once suitable placement has been confirmed in this manner, the proximal end of the feeding tube may be externally secured to the patient in conventional fashion.

FIGS. 11-17 illustrate another embodiment of a feeding tube assembly 100. Feeding tube assembly 100 comprises a feeding tube 102 and a mandrel 120. Echogenic enhancements, such as echogenic bands 118A, 118B, 118C, may be applied to feeding tube distal portion 104, as before. As described herein, this assembly may be inserted into the small intestine, and subjected to real-time ultrasound visualization, in a manner similar to that of the embodiments shown in FIGS. 1-10.

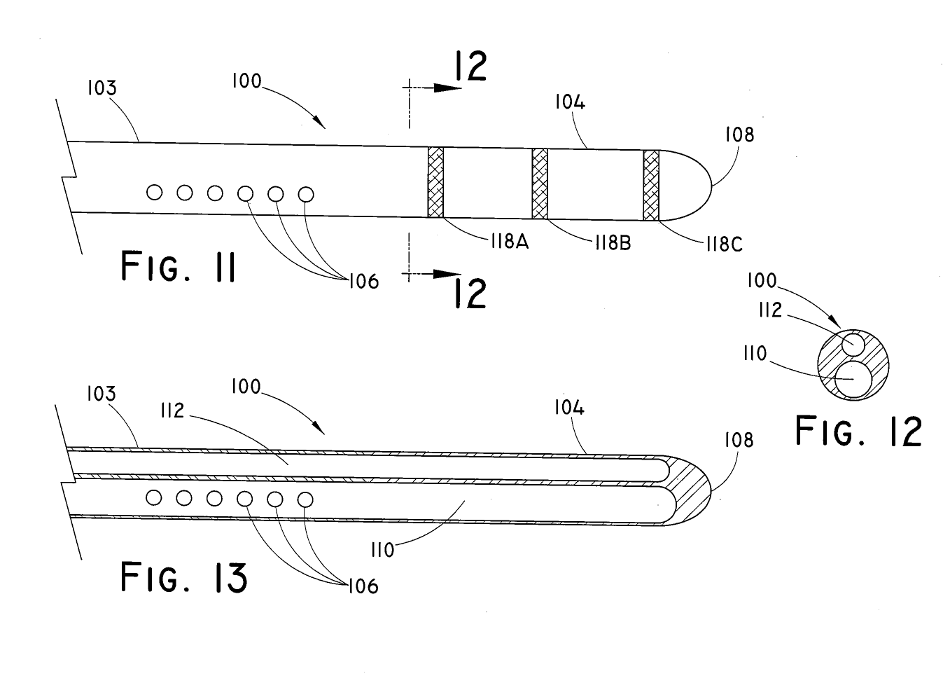

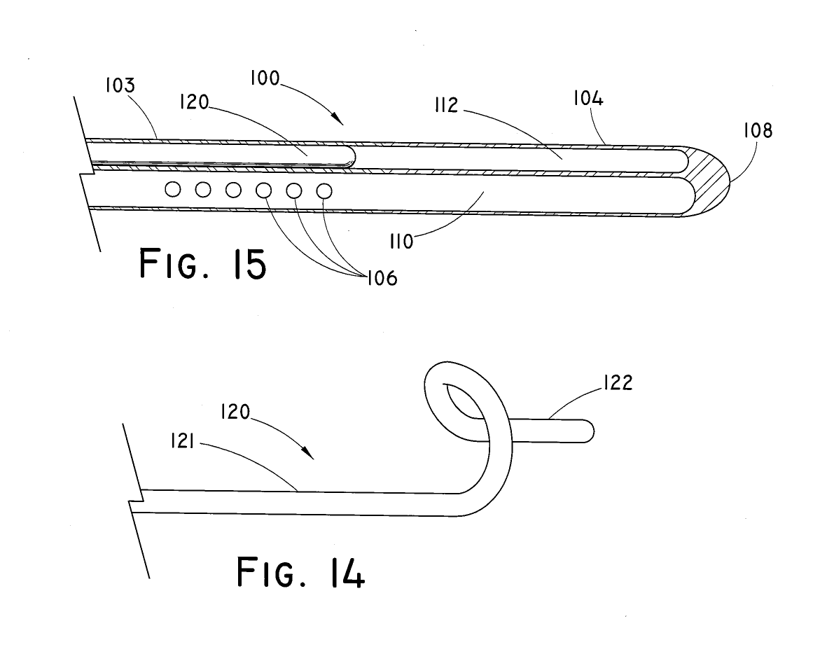

FIG. 11 is a side view of a portion of feeding tube 102. FIG. 12 is a transverse cross-sectional view of feeding tube 102 along line 12-12 of FIG. 11. FIG. 13 is a longitudinal cross-sectional view of feeding tube 102 of FIG. 11. Feeding tube 102 may be a naso-jejunal feeding tube, as described above with reference to FIG. 1. Feeding tube 102 comprises an elongated tubular member having a proximal portion 103 and a distal portion 104. The proximal end of proximal portion 103 is conventional, and is not shown in the figures. Feeding tube 102 may be formed of the same compositions as feeding tube 10 and may have dimensions generally similar to feeding tube 10. As shown in FIGS. 12 and 13, the feeding tube includes lumens 110, 112 extending therethrough. Lumen 110 is sized for transmission of a food product to the patient. Lumen 112 is sized for receiving a mandrel 120, as illustrated in FIGS. 14-16, and as further described below. Although lumens 110, 112 may have various transverse cross-sectional dimensions relative to each other, it is generally preferred to provide lumen 110 with a larger transverse cross-sectional dimension than lumen 112, so that a maximal volume is provided in lumen 110 for fluid flow.

Feeding tube 102 includes one or more feeding tube side ports, or apertures, 106 disposed along the length of the feeding tube to permit passage of nutritional products from lumen 110 into the jejunum in well-known fashion. In this non-limiting embodiment, six ports 106 extend through the feeding tube 102 along each side thereof. Those skilled in the art will appreciate that more, or fewer, side ports may be provided if desired, and that the side ports may be aligned along the feeding tube in any desired arrangement. As with feeding tube 10, feeding tube 102 will preferably taper to a closed distal tip 108.

Distal portion 104 comprises the distalmost length of the tubular member, and preferably, the distalmost 5 to 13 cm of the tubular member. Feeding tube 102 is formed from one or more compositions arranged such that distal portion 104 has a lower durometer (i.e., is more flexible) than proximal portion 103. Those skilled in the art are aware of various methods of forming a feeding tube to have a lower durometer distal portion. For example, the tube can be extruded in a continuous operation such that designated portions of the tube can be of different durometer. With continuous extrusion, a tubular member can be extruded in well-known fashion to be, e.g., rigid or semi-rigid at one end and more flexible at the other end. The tubular member can be extruded to provide a gradual durometer decrease over a defined length of the tube, or can be extruded to provide as many segments of different durometer as desired. Alternatively, a tube having a first durometer can be bonded in well-known fashion (e.g., thermal bonding) to a tube having a second durometer. For optimal bonding, the proximal portion 103 and the distal portion 104 are preferably formed of the same or a similar polymer (e.g., a polyether block amide, such as PEBAX®). However, the proximal portion and distal portion need not necessarily be formed of the same or a similar polymer, as long as they are formed of compositions capable of securely being affixed to each other, e.g., by bonding or adhesion.

FIG. 14 illustrates a stiffening member, or mandrel, 120. FIGS. 15-17 illustrate the assembly 100 comprising the feeding tube 102 and mandrel 120. As shown in FIG. 14, mandrel 120 comprises an elongated rod-like member 121 having a distal end 122 conformable to a desired shape. In this example, mandrel distal end 122 is shaped into a loop, although those skilled in the art will appreciate that the distal end 122 can alternatively be shaped into other configurations to attain the results discussed herein.

Preferably, mandrel 120 is formed of a surgical grade alloy wire, e.g., a shape memory or superelastic wire such as nitinol. Shaped distal end 122 may be straightened as described herein, such that straightened mandrel 120 can be received in tubular member lumen 112, as shown in FIG. 15. Typically, mandrel 120 will have an overall length of at least about 100 cm. As indicated above, the shaped distal end 122 will preferably have a length (when fully straightened) of about 5 to 13 cm. As further discussed below, mandrel 120 is formed of a composition having a durometer lower than the durometer of tubular member proximal portion 103, but having a durometer greater than the durometer of tubular member distal portion 104. Distal end 122 of mandrel 120 can be pre-formed in conventional fashion, e.g., by heat-setting it to the desired loop configuration. When at room temperature, the mandrel should assume a generally linear (e.g., superelastic) orientation.

As stated, the feeding tube is formed of one or more compositions (e.g., a polyurethane, a polyether block amide, etc) wherein the proximal end has a higher durometer than the distal end. If desired, the proximal end of the feeding tube can be reinforced with, e.g., a wire coil or braid as shown in U.S. Pat. No. 5,380,304, incorporated by reference herein, to provide added stiffness such that the mandrel can readily conform to the shape of the proximal portion of the tube. The distal end of the feeding tube will preferably not be reinforced, thereby allowing it to readily take on the pre-determined shape of the mandrel once the mandrel is advanced into the distal end.

In FIG. 15, mandrel 120 is shown extending substantially the length of tubular member proximal portion 103, but not extending into tubular member distal portion 104. Since tubular member proximal portion 103 has a higher durometer (i.e., greater stiffness) than that of mandrel 120, mandrel 120 is not able to overcome the linearity resulting from being received in high durometer tubular member proximal portion 103, and therefore, is not able to revert to its pre-determined shaped condition. Rather, mandrel 120 is maintained in a straightened, generally linear, condition as shown in FIG. 15, or alternatively, any non-linear condition that the proximal portion may have assumed at the time.

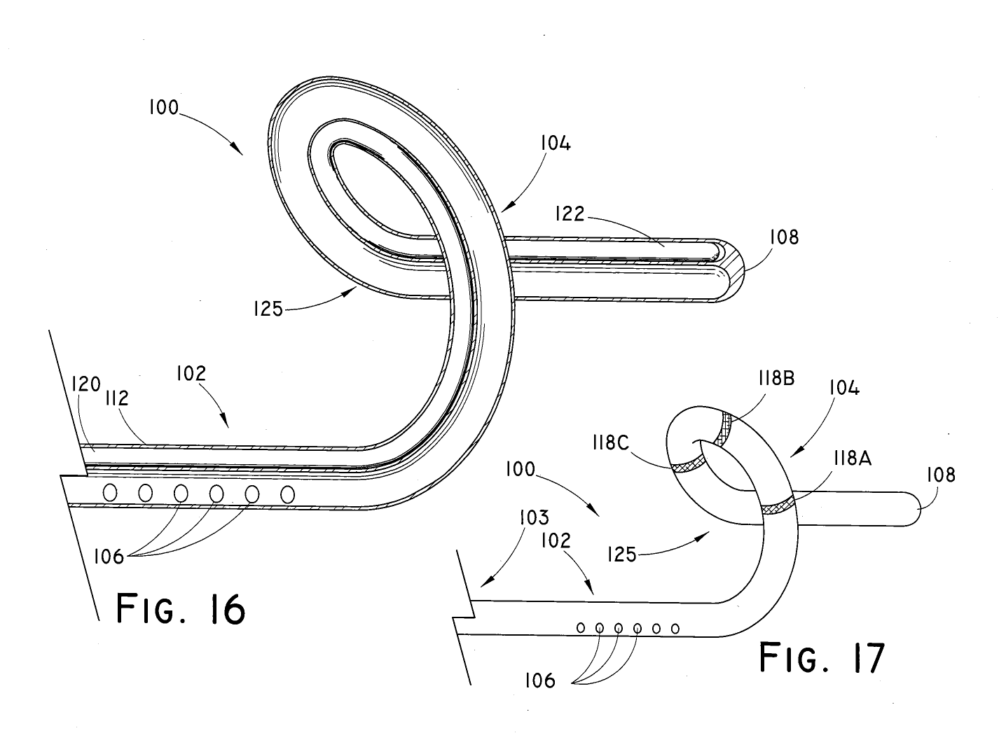

In FIG. 16, mandrel 120 has been further advanced along lumen 112, when compared to the position of the mandrel in FIG. 15, such that the mandrel distal end 122 extends substantially to the end of tubular member distal portion 104. Since the mandrel has a greater durometer than that of tubular member distal portion 104, once mandrel distal end 122 has been released from the constraints of tubular member proximal portion 103, the mandrel distal end is able to revert to its shaped configuration. As a result, since the durometer of the mandrel is greater than that of lower durometer tubular member distal portion 104, the distal portion takes on the shaped configuration of mandrel distal end 122, as shown in FIGS. 16 and 17. In the non-limiting embodiment shown, the shaped configuration comprises a loop 125 at the distal end of the tubular member.

Echogenic enhancing features are provided along tubular member distal portion 104. In the non-limiting embodiment of FIGS. 11-17, the echogenic features comprise three echogenic bands 118A, 1186, 118C spaced along distal portion 104. Once again, this number and type of echogenic enhancement is only one example of numerous possible variations of number, and kind, of echogenic enhancements that may be incorporated into the tubular member to enhance visualization under ultrasound. In this case, bands 118A, 1186, and 118C are spaced along the length of tubular member distal portion 104 such that they are spaced approximately 120 degrees along the resulting loop 125.

Those skilled in the art will appreciate that feeding tube assembly 100 may be advanced into the small intestine, and arranged therein to provide real-time ultrasound visualization in a manner generally similar to that shown and described hereinabove with reference to FIGS. 7-10. Initially, mandrel 120 is inserted into the proximal end of feeding tube 102, and advanced substantially to the end of proximal, or high durometer, portion 103. At this time, high durometer proximal portion 103 maintains mandrel 120 in the elongated position shown in FIG. 15. The distal end of the feeding tube is then advanced into the jejunum as described above, e.g., utilizing peristaltic contractions. When it is desired to view the placement of the feeding tube, mandrel distal end 122 is further advanced into the distal, or lower durometer, portion 104 of the feeding tube. As stated above, since lower durometer distal portion 104 has insufficient stiffness to maintain mandrel distal end 122 in the elongated condition shown in FIG. 15, mandrel end 122 then reverts to the shaped configuration shown in the figures, thereby causing distal portion 104 of the feeding tube to form a loop 125, as shown in FIGS. 16 and 17.

At this time, the ultrasound transducer head can be aligned externally of the patient as described above, and a real-time ultrasound image is obtained. If the quality of the image is not deemed suitable, the clinician can simply further advance the assembly along the small intestine, and obtain another ultrasound image.

While these features have been disclosed in connection with the illustrated preferred embodiments, other embodiments of the invention will be apparent to those skilled in the art that come within the spirit of the invention as defined in the following claims.

1. A tube for insertion into a body passageway of a patient, comprising:
a generally elongated tubular member having a proximal portion, a distal portion, a lumen extending between said proximal portion and said distal portion, and at least one aperture at said distal portion, said aperture sized and positioned for passage of fluid material therethrough from said lumen to a target area in said body passageway, said tubular member being structured such that said distal portion is selectively movable between said generally elongated configuration and a shaped configuration, a length of said distal portion comprising an echogenic capability such that said distal portion length is visible under ultrasound visualization in said shaped configuration.

2. The tube of claim 1, comprising:
a stiffening member removably receivable along said tubular member proximal portion and distal portion, said stiffening member having a stiffness relative to a stiffness of said tubular member distal portion such that said distal portion comprises said generally elongated configuration when said stiffening member is received therealong, said tubular member distal portion movable to said shaped configuration upon a withdrawal of said stiffening member along said distal portion; and wherein said echogenic capability comprises at least one echogenic member disposed along said shaped configuration.

3. The tube of claim 2, wherein said lumen comprises a first lumen, said elongated tubular member further comprising a second lumen, said stiffening member being removably receivable in said second lumen.

4. The tube of claim 2, wherein said tubular member comprises an internal memory for movement to said shaped configuration upon a withdrawal of said stiffening member along said tubular member.

5. The tube of claim 1, wherein said shaped configuration comprises a loop, and wherein said echogenic capability comprises one or more echogenic members disposed along said loop.

6. The tube of claim 5, wherein said echogenic members comprise respective bands disposed along said loop, said bands comprising deformations disposed along a surface thereof.

7. The tube of claim 5, wherein said one or more echogenic members comprise a pair of echogenic members, said echogenic members initially disposed along said distal portion of said elongated tubular member, and arranged thereat such that said echogenic members are substantially disposed at diametrically opposing sides of said loop.

8. The tube of claim 1, wherein said echogenic capability comprises an echogenic member disposed along said shaped configuration, said echogenic member comprising deformations thereon.

9. The tube of claim 1, comprising:
a tension member engaged with said tubular member distal portion, said tension member arranged along said tubular member in a manner to selectively draw said distal portion into said shaped configuration.

10. A method for positioning a feeding tube in the jejunum of a patient, comprising:
inserting a distal end of a feeding tube into an oral cavity of a patient, the feeding tube comprising a generally elongated tubular member having a proximal portion, a distal portion having an echogenic surface, a lumen extending between said proximal portion and said distal portion, at least one aperture at said distal portion for passage of fluid material from said lumen to said jejunum, and further comprising a stiffening member extending along said tubular member proximal portion and said distal portion, said tubular member being selectively maneuverable between said generally elongated condition when said stiffening member extends therealong and a shaped condition along said distal portion when said stiffening member is withdrawn from said distal portion;
advancing said feeding tube distal end through the stomach of the patient such that said distal portion of said generally elongated tubular member extends into the small intestine of the patient;
withdrawing said stiffening member from said distal portion such that said distal portion maneuvers into said shaped condition; and
viewing a placement of said shaped distal portion via ultrasound visualization of said echogenic surface.

11. The method of claim 10, wherein said shaped condition comprises a loop configured to at least substantially span opposing walls of said small intestine.

12. The method of claim 11, wherein said echogenic surface comprises at least one echogenic member disposed along said distal portion.

13. The method of claim 12, wherein said echogenic surface comprises a pair of echogenic members spaced along said distal portion such that said echogenic members are substantially disposed at diametrically opposite sides of said loop.

14. The method of claim 12, wherein said at least one echogenic member comprises a band having deformations along an outer surface thereof.

15. The method of claim 11, further comprising the steps of:
advancing said feeding tube along said small intestine following said viewing; and
viewing a placement of said shaped distal portion in the jejunum portion of the small intestine.

16. The method of claim 10, wherein the feeding tube is advanced such that said tubular member distal portion extends into the jejunum, and said ultrasound visualization confirms placement of said distal portion in the jejunum.

17. A tube for insertion into a body passageway of a patient, comprising:
a generally elongated tubular member having a proximal portion, a distal portion, a pair of lumens extending between said proximal portion and said distal portion, and at least one aperture along a length of said tubular member, said aperture sized and positioned for passage of fluid material therethrough from a first one of said lumens to a target area in said body passageway, said proximal portion of said tubular member having a higher stiffness, and said distal portion of said tubular member having a lower stiffness, said distal portion having an echogenic material disposed therealong; and
a mandrel slidably received in a second one of said lumens, said mandrel having a stiffness less than a stiffness of said tubular member proximal portion, and having a stiffness greater than a stiffness of said tubular member distal portion, said mandrel structured such that a distal length thereof has a tendency to assume a shaped configuration in an absence of restraint thereupon, such that when said mandrel distal length is received along said tubular member proximal portion having said higher stiffness said mandrel distal length comprises said generally elongated condition of said tubular member proximal portion, and when said mandrel distal length is received along said tubular member distal portion having said lower stiffness said mandrel distal length and said tubular member distal portion having said echogenic material disposed therealong assume said shaped configuration.

18. The tube of claim 17, wherein said shaped configuration comprises a loop, and said echogenic material comprises an echogenic member disposed along said loop.

19. The tube of claim 17, wherein said shaped configuration comprises a loop, and said echogenic material comprises a plurality of echogenic bands disposed along said loop.

20. The tube of claim 19, wherein said echogenic bands include deformations formed on a surface therefore capable of echogenic members comprise respective bands disposed along said loop, said bands comprising deformations disposed along an exterior surface thereof capable of scattering ultrasound energy.
